(19) United States
(12) Patent Application Publication
(10) Pub. No.: US 2022/0375626 A1
(43) Pub. Date: Nov. 24, 2022

(54) Telehealth System and Method

(71) Applicant: Nuance Communications, Inc., Burlington, MA (US)

(72) Inventors: Rong GONG, Vienna (AT); Ljubomir Milanovic, Vienna (AT); Kaarel Kaljurand, Vienna (AT)

(21) Appl. No.: 17/326,600

(22) Filed: May 21, 2021

(51) Int. Cl. G16H 80/00; G06F 40/58; G16H 40/67

(52) U.S. Cl. CPC G16H 80/00; G06F 40/58; G16H 40/67

(57) ABSTRACT

A method, computer program product, and computing system for: monitoring a conversation between a plurality of participants of a telehealth session; identifying an addressable issue within the conversation; and initiating an action to mitigate the addressable issue.

### TECHNICAL FIELD

This disclosure relates to telehealth systems and methods and, more particularly, to telehealth systems and methods that confirm a party's understanding of a conversation.

### BACKGROUND

Regular access to medical professionals is of paramount importance for people maintaining their health. Accordingly, it has been shown that annual physicals are instrumental to maintaining a patient's health. Further and in the event of an illness, it is important that a medical professional is engaged in a timely fashion.

Unfortunately, such medical professionals are often not timely engaged. For example, it is often difficult for people to travel to see a doctor. Further and especially when a specialist is needed, a significant amount of travel may be required in order to visit with such a medical professional. While telehealth visits may solve some of these issues by allowing a patient to visit with a doctor without having to actually travel to the doctor's office, a lack of understanding may exist between the medical professional and the patient due to the remote nature of such telehealth visits.

### SUMMARY OF DISCLOSURE

In one implementation, a computer-implemented method is executed on a computing system and includes: monitoring a conversation between a plurality of participants of a telehealth session; identifying an addressable issue within the conversation; and initiating an action to mitigate the addressable issue.

One or more of the following features may be included. A first participant of the plurality of participants may be a medical professional. A second participant of the plurality of participants may be a patient. The addressable issue may be a potential language barrier with one of the participants of the telehealth session. Initiating an action to mitigate the addressable issue may include: translating audio received by one of the participants of the telehealth session from a first language to a second language. Translating audio received by one of the participants of the telehealth session from a first language to a second language may include: providing a written translation of audio received by one of the participants of the telehealth session from the first language to the second language. Translating audio received by one of the participants of the telehealth session from a first language to a second language may include: providing a verbal translation of audio received by one of the participants of the telehealth session from the first language to the second language. The addressable issue may be a potential lack of understanding with one of the participants of the telehealth session. Initiating an action to mitigate the addressable issue may include: providing a text-based summary of at least a portion of the conversation to a specific participant of the telehealth session; and requesting that the specific participant of the healthcare session confirm their understanding of the text-based summary. The text-based summary may be a text-based summary of at least a portion of the conversation provided by the specific participant of the healthcare session. The text-based summary may be a text-based summary of at least a portion of the conversation provided by another participant of the healthcare session.

In another implementation, a computer program product resides on a computer readable medium and has a plurality of instructions stored on it. When executed by a processor, the instructions cause the processor to perform operations including: monitoring a conversation between a plurality of participants of a telehealth session; identifying an addressable issue within the conversation; and initiating an action to mitigate the addressable issue.

One or more of the following features may be included. A first participant of the plurality of participants may be a medical professional. A second participant of the plurality of participants may be a patient. The addressable issue may be a potential language barrier with one of the participants of the telehealth session. Initiating an action to mitigate the addressable issue may include: translating audio received by one of the participants of the telehealth session from a first language to a second language. Translating audio received by one of the participants of the telehealth session from a first language to a second language may include: providing a written translation of audio received by one of the participants of the telehealth session from the first language to the second language. Translating audio received by one of the participants of the telehealth session from a first language to a second language may include: providing a verbal translation of audio received by one of the participants of the telehealth session from the first language to the second language. The addressable issue may be a potential lack of understanding with one of the participants of the telehealth session. Initiating an action to mitigate the addressable issue may include: providing a text-based summary of at least a portion of the conversation to a specific participant of the telehealth session; and requesting that the specific participant of the healthcare session confirm their understanding of the text-based summary. The text-based summary may be a text-based summary of at least a portion of the conversation provided by the specific participant of the healthcare session. The text-based summary may be a text-based summary of at least a portion of the conversation provided by another participant of the healthcare session.

In another implementation, a computing system includes a processor and memory is configured to perform operations including: monitoring a conversation between a plurality of participants of a telehealth session; identifying an addressable issue within the conversation; and initiating an action to mitigate the addressable issue.

One or more of the following features may be included. A first participant of the plurality of participants may be a medical professional. A second participant of the plurality of participants may be a patient. The addressable issue may be a potential language barrier with one of the participants of the telehealth session. Initiating an action to mitigate the addressable issue may include: translating audio received by one of the participants of the telehealth session from a first language to a second language. Translating audio received by one of the participants of the telehealth session from a first language to a second language may include: providing a written translation of audio received by one of the participants of the telehealth session from the first language to the second language. Translating audio received by one of the participants of the telehealth session from a first language to a second language may include: providing a verbal translation of audio received by one of the participants of the telehealth session from the first language to the second language. The addressable issue may be a potential lack of understanding with one of the participants of the telehealth session. Initiating an action to mitigate the addressable issue may include: providing a text-based summary of at least a portion of the conversation to a specific participant of the telehealth session; and requesting that the specific participant of the healthcare session confirm their understanding of the text-based summary. The text-based summary may be a text-based summary of at least a portion of the conversation provided by the specific participant of the healthcare session. The text-based summary may be a text-based summary of at least a portion of the conversation provided by another participant of the healthcare session.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a telehealth system and a telehealth process coupled to a distributed computing network;

FIG. 2 is a diagrammatic view of the telehealth system of FIG. 1;

FIG. 3 is a flow chart of the telehealth process of FIG. 1; and

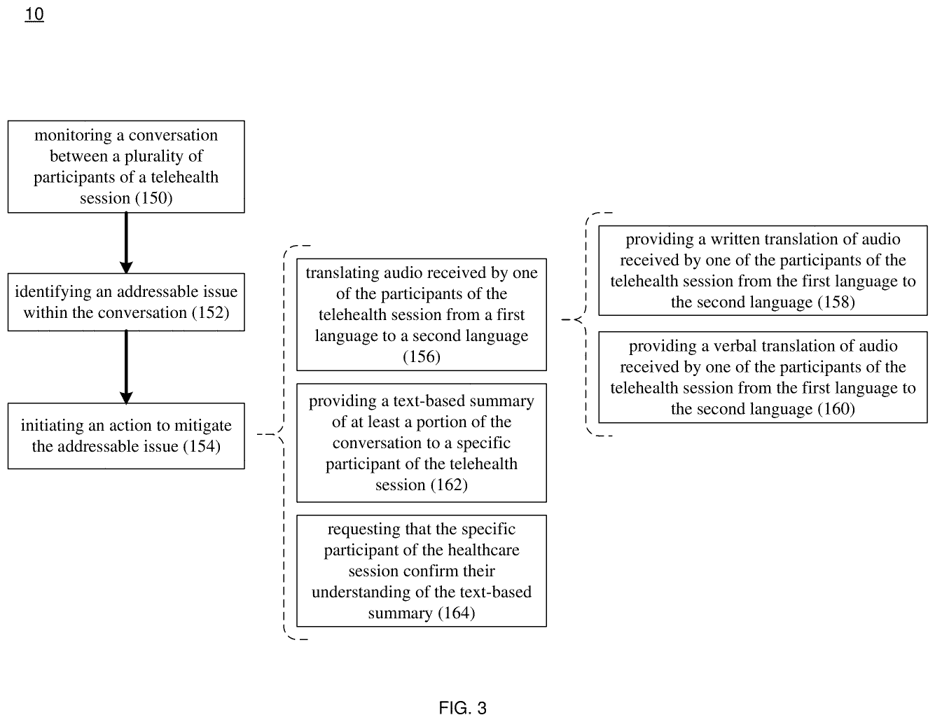

FIG. 4 is a diagrammatic view of the telehealth system of FIG. 1.

Like reference symbols in the various drawings indicate like elements.

### DETAILED DESCRIPTION OF THE PREFERRED EMBODIMENTS

### System Overview

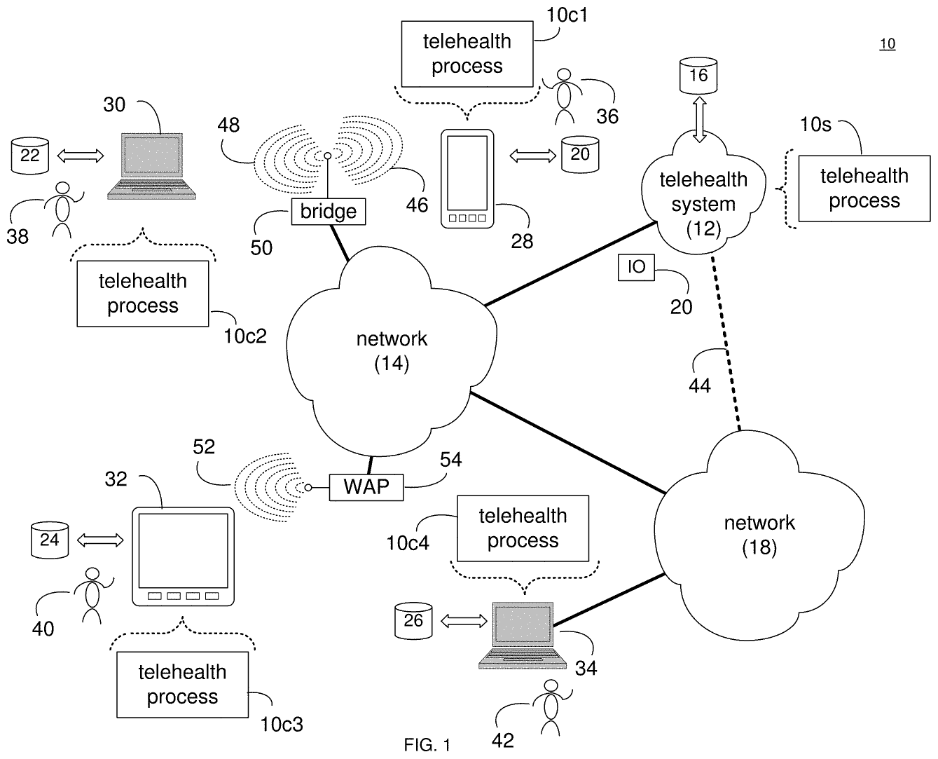

Referring to FIG. 1, there is shown telehealth process 10. As will be discussed below in greater detail, telehealth process 10 may be configured to effectuate a telehealth visit between a medical professional and a patient.

Telehealth process 10 may be implemented as a server-side process, a client-side process, or a hybrid server-side/client-side process. For example, telehealth process 10 may be implemented as a purely server-side process via telehealth process 10s. Alternatively, telehealth process 10 may be implemented as a purely client-side process via one or more of telehealth process 10c1, telehealth process 10c2, telehealth process 10c3, and telehealth process 10c4. Alternatively still, telehealth process 10 may be implemented as a hybrid server-side/client-side process via telehealth process 10s in combination with one or more of telehealth process 10c1, telehealth process 10c2, telehealth process 10c3, and telehealth process 10c4.

Accordingly, telehealth process 10 as used in this disclosure may include any combination of telehealth process 10s, telehealth process 10c1, telehealth process 10c2, telehealth process 10c3, and telehealth process 10c4.

Telehealth process 10s may be a server application and may reside on and may be executed by telehealth system 12, which may be connected to network 14 (e.g., the Internet or a local area network). Telehealth system 12 may include various components, examples of which may include but are not limited to: a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, one or more software applications, one or more software platforms, a cloud-based computational system, and a cloud-based storage platform.

As is known in the art, a SAN may include one or more of a personal computer, a server computer, a series of server computers, a mini computer, a mainframe computer, a RAID device and a NAS system. The various components of telehealth system 12 may execute one or more operating systems, examples of which may include but are not limited to: Microsoft Windows Server™; Red Hat Linux Unix™, or a custom operating system, for example.

The instruction sets and subroutines of telehealth process 10s, which may be stored on storage device 16 coupled to telehealth system 12, may be executed by one or more processors (not shown) and one or more memory architectures (not shown) included within telehealth system 12. Examples of storage device 16 may include but are not limited to: a hard disk drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Various IO requests (e.g. IO request 20) may be sent from telehealth process 10s, telehealth process 10c1, telehealth process 10c2, telehealth process 10c3 and/or telehealth process 10c4 to telehealth system 12. Examples of IO request 20 may include but are not limited to data write requests (i.e. a request that content be written to data acquisition system 12) and data read requests (i.e. a request that content be read from data acquisition system 12).

The instruction sets and subroutines of telehealth process 10c1, telehealth process 10c2, telehealth process 10c3 and/or telehealth process 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Storage devices 20, 22, 24, 26 may include but are not limited to: hard disk drives; optical drives; RAID devices; random access memories (RAM); read-only memories (ROM), and all forms of flash memory storage devices.

Examples of client electronic devices 28, 30, 32, 34 may include, but are not limited to, data-enabled, cellular telephone 28, laptop computer 30, tablet computer 32, laptop computer 34, a notebook computer (not shown), a server computer (not shown), a gaming console (not shown), a smart television (not shown), and a dedicated network device (not shown). Client electronic devices 28, 30, 32, 34 may each execute an operating system, examples of which may include but are not limited to Microsoft Windows™, Android™, webOS™, iOS™, Red Hat Linux™, or a custom operating system.

Users 36, 38, 40, 42 may access telehealth process 10 directly through network 14 or through secondary network 18. Further, telehealth process 10 may be connected to network 14 through secondary network 18, as illustrated with link line 44.

The various client electronic devices (e.g., client electronic devices 28, 30, 32, 34) may be directly or indirectly coupled to network 14 (or network 18). For example, data-enabled, cellular telephone 28 and laptop computer 30 are shown wirelessly coupled to network 14 via wireless communication channels 46, 48 (respectively) established between data-enabled, cellular telephone 28, laptop computer 30 (respectively) and cellular network/bridge 50, which is shown directly coupled to network 14. Further, tablet computer 32 is shown wirelessly coupled to network 14 via wireless communication channel 52 established between tablet computer 32 and wireless access point (i.e., WAP) 54, which is shown directly coupled to network 14. WAP 54 may be, for example, an IEEE 802.11a, 802.11b, 802.11g, 802.11n, Wi-Fi, and/or Bluetooth device that is capable of establishing wireless communication channel 52 between tablet computer 32 and WAP 54. Additionally, laptop computer 34 is shown directly coupled to network 18 via a hardwired network connection.

### The Telehealth System

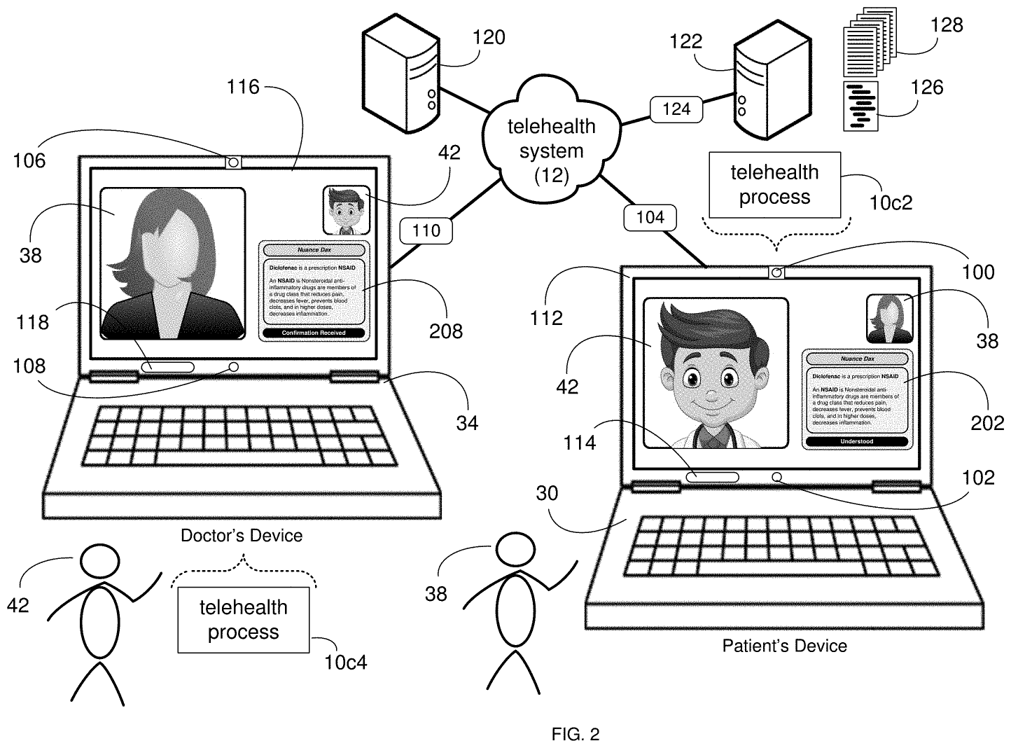

Referring also to FIG. 2 and as discussed above, telehealth process 10 in combination with telehealth system 12 may be configured to enable telehealth sessions between a medical professional and a patient. While the following illustrative example concerns a telehealth session between a single doctor and a single patient, this is for illustrative purposes only and is not intended to be a limitation of this disclosure, as other configurations are possible and are considered to be within the scope of this disclosure. For example, telehealth process 10 in combination with telehealth system 12 may establish a telehealth session between a team of medical professionals, a patient and one or more interested parties (e.g., patients, relatives, guardians, etc.).

As is known in the art, telehealth is the distribution of health-related services and information via electronic information and telecommunication technologies. Telehealth sessions may allow long-distance patient and clinician contact, care, advice, reminders, education, intervention, monitoring, and remote admissions. Telemedicine may sometimes be used as a synonym, or may be used in a more limited sense to describe remote clinical services, such as diagnosis and monitoring. When rural settings, lack of transport, a lack of mobility, decreased funding, or a lack of staff restrict access to care; telehealth may bridge the gap, as well as provide: distance-learning, meetings, supervision, and presentations between practitioners; online information and health data management; and healthcare system integration. Telehealth may include two clinicians discussing a case over video conference; a robotic surgery occurring through remote access; physical therapy performed via digital monitoring instruments, live feed and application combinations; tests being forwarded between facilities for interpretation by a higher specialist; home monitoring through continuous sending of patient health data; client to practitioner online conference; or even videophone interpretation during a consult.

For example, assume that a patient (e.g., user 38) wishes to meet with a doctor (e.g., user 42). However and for various reasons (e.g., a pandemic, a geographic distance, lack of geographically-proximate practitioners, etc.), the patient (e.g., user 38) cannot physically meet with the doctor (e.g., user 42), Therefore, telehealth process 10 in combination with telehealth system 12 may enable the patient (e.g., user 38) to virtually meet with the doctor (e.g., user 42) via a telehealth session.

Specifically, the patient (e.g., user 38) may utilize their client electronic device (e.g., laptop computer 30) and telehealth process 10c2 to participate in a telehealth session with the doctor (e.g., user 42), who will utilize their client electronic device (e.g., laptop computer 34) and telehealth process 10c4.

The client electronic device (e.g., laptop computer 30) of the patient (e.g., user 38) may include video camera 100 and microphone 102 to generate audio/video stream (e.g., AV stream 104) that may be provided via telehealth system 12 to the client electronic device (e.g., laptop computer 34) of the doctor (e.g., user 42). Additionally, the client electronic device (e.g., laptop computer 34) of the doctor (e.g., user 42) may include video camera 106 and microphone 108 to generate audio/video stream (e.g., AV stream 110) that may be provided via telehealth system 12 to the client electronic device (e.g., laptop computer 30) of the patient (e.g., user 38).

Telehealth process 10 (generally) and telehealth process 10c2 (specifically) may render user interface 112 on the client electronic device (e.g., laptop computer 30) of the patient (e.g., user 38). User interface 112 may render a smaller-size video feed of the patient (e.g., user 38), which is obtained from video camera 100 and allows the patient (e.g., user 38) to monitor the way that they appear within the telehealth session. Further, user interface 112 may render a larger-size video feed of the doctor (e.g., user 42) as well as an audio signal from the doctor (e.g., user 42) on speaker assembly 114, both of which are obtained from AV stream 110 and allow the patient (e.g., user 38) to see, hear and interact with the doctor (e.g., user 42).

Telehealth process 10 (generally) and telehealth process 10c4 (specifically) may render user interface 116 on the client electronic device (e.g., laptop computer 34) of the doctor (e.g., user 42). User interface 116 may render a smaller-size video feed of the doctor (e.g., user 42), which is obtained from video camera 106 and allows the doctor (e.g., user 42) to monitor the way that they appear within the telehealth session. Further, user interface 116 may render a larger-size video feed of the patient (e.g., user 38) as well as an audio signal from the patient (e.g., user 38) on speaker assembly 118, both of which are obtained from AV stream 104 and allow the doctor (e.g., user 42) to see, hear and interact with the patient (e.g., user 38).

Telehealth system 12 may be coupled to one or more collaborating systems (e.g., collaborating system 120), examples of which may include but are not limited to a collaborating system executing a PACS system and a collaborating system executing an EHR system.
As is known in the art, a PACS (Picture Archiving and Communication System) system is a medical imaging technology that provides economical storage and convenient access to images from multiple modalities (source machine types). Electronic images and reports may be transmitted digitally via PACS; thus eliminating the need to manually file, retrieve and/or transport film jackets. The universal format for PACS image storage and transfer is DICOM (Digital Imaging and Communications in Medicine). Non-image data, such as scanned documents, may be incorporated using consumer industry standard formats like PDF (Portable Document Format), once encapsulated in DICOM.
As is known in the art, an EHR (Electronic Health Record) system is a systematized collection of patient and population electronically stored health information in a digital format. These records may be shared across different health care settings, wherein records maybe shared through network-connected, enterprise-wide information systems or other information networks and exchanges. An EHR system may define a range of data, including demographics, medical histories, medications and allergies, immunization status, laboratory test results, radiology images, vital signs, personal statistics, and billing information.

Accordingly and through the use of such a PACS system and an EHR system, the doctor (e.g., user 42) may access and review medical images and electronic health records associated with the patient (e.g., user 38).

Telehealth system 12 may also be coupled to an ambient cooperative intelligence system (e.g., ACI system 122), an example of which is the Dragon Ambient eXperience (DAX) system offered by Nuance of Burlington, MA. ACI system 122 may be configured to automate the collection and processing of clinical encounter information (e.g., encounter information 124) associated with a patient encounter (e.g., the telehealth session between the patient (e.g., user 38) and the doctor (e.g., user 42)). For example, ACI system 122 may be configured to process the encounter information (e.g., encounter information 124) to generate an encounter transcript (e.g., encounter transcript 126), wherein ACI system 122 may then process at least a portion of the encounter transcript (e.g., encounter transcript 126) to populate at least a portion of a medical record (e.g., medical record 128) associated with the patient encounter (e.g., the telehealth session between the patient (e.g., user 38) and the doctor (e.g., user 42)). Accordingly, ACI system 122 may collect clinical encounter information (e.g., encounter information 124) associated with the telehealth session between the patient (e.g., user 38) and the doctor (e.g., user 42) to generate/store/distribute medical records (e.g., medical records 128) for the patient (e.g., user 38).

ACI system 122 may utilize natural language processing and artificial intelligence to process data included within the encounter transcript (e.g., encounter transcript 126) and/or data included within AV streams 104, 110. For example and as will be discussed below, ACI system 122 may monitor the conversation between the patient (e.g., user 38) and the doctor (e.g., user 42) during the above-described telehealth session to identify issues that should be addressed.

### The Telehealth Process

Telehealth process 10 may monitor 150 a conversation between a plurality of participants of a telehealth session. An example of such a telehealth session may include the above-described telehealth session between the patient (e.g., user 38) and the doctor (e.g., user 42). Accordingly, a first participant of the plurality of participants may be a medical professional (e.g., user 42) and a second participant of the plurality of participants may be a patient (e.g., user 38).

When monitoring 150 the conversation between (in this example) the patient (e.g., user 38) and the doctor (e.g., user 42), natural language processing and artificial intelligence may be utilized to process data included within the encounter transcript (e.g., encounter transcript 126) and/or data included within AV streams 104, 110.

Telehealth process 10 may identify 152 an addressable issue within the conversation between (in this example) the patient (e.g., user 38) and the doctor (e.g., user 42). As will be discussed below, examples of such addressable issues may include but are not limited to a situation in which one party of the conversation may not fully grasp or understand the subject of the conversation.

In the event that such an addressable issue within the conversation is identified 152, telehealth process 10 may initiate 154 an action to mitigate the addressable issue. As will be discussed below, the particular type of action initiated 154 to mitigate the addressable issue may vary depending upon the addressable issue identified 152.

For the following example, assume that the addressable issue identified 152 may be a potential language barrier with one of the participants of the telehealth session. Specifically, assume that telehealth process 10 determines that the doctor (e.g., user 42) is a native English speaker, while the patient (e.g., user 38) is a native Spanish speaker. When making such a determination, telehealth process 10 may simply make an inquiry concerning the native language of the patient (e.g., user 38) and the doctor (e.g., user 42). This inquiry may be made by e.g., reviewing a profile of (in this example) the patient (e.g., user 38) and the doctor (e.g., user 42) and/or proactively asking about the native language of the patient (e.g., user 38) and the doctor (e.g., user 42). Additionally/alternatively, telehealth process 10 may analyze the accents of the participants of the telehealth session in an attempt to determine the native languages of the participants. In the event that the participants of the telehealth session (e.g., the patient (e.g., user 38) and the doctor (e.g., user 42)) have differing native languages, a potential language barrier situation may exist.

Accordingly and in the situation where the addressable issue identified 152 is a potential language barrier, when initiating 154 an action to mitigate the addressable issue, telehealth process 10 may translate 156 audio received by one of the participants of the telehealth session from a first language to a second language. Accordingly and for the situation in which the doctor (e.g., user 42) is a native English speaker and the patient (e.g., user 38) is a native Spanish speaker, telehealth process 10 may translate 156 the audio received by the patient (e.g., user 38) from English (e.g., the native language of the doctor (e.g., user 42)) to Spanish (e.g., the native language of the patient (e.g., user 38)).

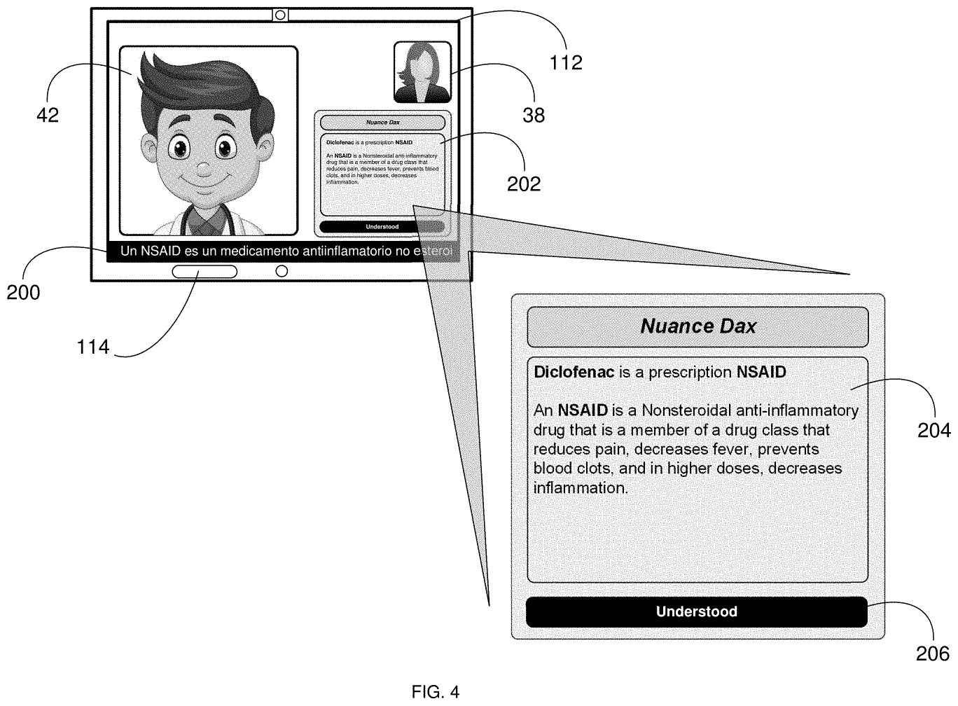

When translating 156 audio received by one of the participants of the telehealth session from a first language to a second language (e.g., from English to Spanish), telehealth process 10 may provide 158 a written translation of audio received by one of the participants of the telehealth session from the first language to the second language. For example and referring also to FIG. 4, telehealth process 10 may render a chyron (e.g., chyron 200) along e.g., a lower edge of user interface 112 that contains (in this example) the English language translation of the audio received from the doctor (e.g., user 42) in a fashion similar to automatically-generated real-time subtitles.

Additionally/alternatively and when translating 156 audio received by one of the participants of the telehealth session from a first language to a second language (e.g., from English to Spanish), telehealth process 10 may provide 160 a verbal translation of audio received by one of the participants of the telehealth session from the first language to the second language. For example, telehealth process 10 may render (on speaker assembly 114) an audio-based translation of the audio received from the doctor (e.g., user 42).

For the following example, assume that the addressable issue identified 152 may be a potential lack of understanding with one of the participants of the telehealth session. Specifically, assume that telehealth process 10 determines that the doctor (e.g., user 42) is providing critical and/or complex information to the patient (e.g., user 38). Examples of such critical and/or complex information may include the doctor (e.g., user 42) providing information to the patient (e.g., user 38) concerning a particular medicine being prescribed to the patient (e.g., user 38) and/or a particular procedure being recommended to the patient (e.g., user 38). When making such a determination, telehealth process 10 may utilize natural language processing and artificial intelligence to process data included within the encounter transcript (e.g., encounter transcript 126) and/or data included within AV streams 104, 110 to determine if critical and/or complex information is being discussed with the patient (e.g., user 38). In the event that telehealth process 10 determines that the doctor (e.g., user 42) is providing critical and/or complex information to the patient (e.g., user 38), telehealth process 10 may provide the patient (e.g., user 38) with additional/supplemental information concerning the critical and/or complex information.

For the following example, assume that the doctor (e.g., user 42) is going to write a prescription to the patient (e.g., user 38) for Diclofenac (a type of NSAID), which telehealth process 10 determines is critical and/or complex information. Accordingly and in the situation where the addressable issue identified 152 is critical and/or complex information, when initiating 154 an action to mitigate the addressable issue, telehealth process 10 may render supplemental information window 202.

Further and when initiating 154 an action to mitigate the addressable issue, telehealth process 10 may provide 162 a text-based summary (e.g., text-based summary 204) of at least a portion of the conversation to a specific participant of the telehealth session. This text-based summary (e.g., text-based summary 204) may be a text-based summary of at least a portion of the conversation provided by another participant of the healthcare session, such as the doctor (e.g., user 42). For example and within supplemental information window 202 rendered on laptop computer 30 of the patient (e.g., user 38), telehealth process 10 may provide 162 text-based summary 204 that states:

### Diclofenac is a prescription NSAID

An NSAID is a Nonsteroidal anti-inflammatory drug that is a member of a drug class that reduces pain, decreases fever, prevents blood clots, and in higher doses, decreases inflammation

When initiating 154 an action to mitigate the addressable issue, telehealth process 10 may request 164 that the specific participant of the healthcare session confirm their understanding of the text-based summary (e.g., text-based summary 204). For example and when rendering supplemental information window 202, telehealth process 10 may render confirmation button 206, wherein the specific participant (e.g., user 38) of the healthcare session may confirm their understanding of text-based summary 204 by clicking on confirmation button 206. Upon the patient (e.g., user 38) confirming their understanding of text-based summary 204, the doctor (e.g., user 42) may be notified of the confirmation by the patient (e.g., user 38) on supplemental information window 208 rendered of laptop computer 34 of the doctor (e.g., user 42).

While the above discussion concerns the text-based summary (e.g., text-based summary 204) being a text-based summary of at least a portion of the conversation provided by another participant of the healthcare session, such as the doctor (e.g., user 42), this is for illustrative purposes only and is not intended to be a limitation of this disclosure, as other configurations are possible and are considered to be within the scope of this disclosure. For example, the text-based summary (e.g., text-based summary 204) may be a text-based summary of at least a portion of the conversation provided by the specific participant of the healthcare session, such as the patient (e.g., user 38). For example, suppose the patient (e.g., user 38) is explaining to the doctor (e.g., user 42) all of the medications that they are currently taking. After making such an explanation, telehealth process 10 may provide 162 a text-based summary (e.g., text-based summary 204) of (in this example) the medications that the patient (e.g., user 38) is currently taking, wherein the patient (e.g., user 38) may subsequently confirm the same.

### General:

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing system, comprising:
monitoring a conversation between a plurality of participants of a telehealth session;
identifying an addressable issue within the conversation; and
initiating an action to mitigate the addressable issue.

2. The computer-implemented method of claim 1 wherein:
a first participant of the plurality of participants is a medical professional; and
a second participant of the plurality of participants is a patient.

3. The computer-implemented method of claim 1 wherein the addressable issue is a potential language barrier with one of the participants of the telehealth session.

4. The computer-implemented method of claim 3 wherein initiating an action to mitigate the addressable issue includes:
translating audio received by one of the participants of the telehealth session from a first language to a second language.

5. The computer-implemented method of claim 4 wherein translating audio received by one of the participants of the telehealth session from a first language to a second language includes:
providing a written translation of audio received by one of the participants of the telehealth session from the first language to the second language.

6. The computer-implemented method of claim 4 wherein translating audio received by one of the participants of the telehealth session from a first language to a second language includes:
providing a verbal translation of audio received by one of the participants of the telehealth session from the first language to the second language.

7. The computer-implemented method of claim 1 wherein the addressable issue is a potential lack of understanding with one of the participants of the telehealth session.

8. The computer-implemented method of claim 7 wherein initiating an action to mitigate the addressable issue includes:
providing a text-based summary of at least a portion of the conversation to a specific participant of the telehealth session; and
requesting that the specific participant of the healthcare session confirm their understanding of the text-based summary.

9. The computer-implemented method of claim 8 wherein the text-based summary is a text-based summary of at least a portion of the conversation provided by the specific participant of the healthcare session.

10. The computer-implemented method of claim 8 wherein the text-based summary is a text-based summary of at least a portion of the conversation provided by another participant of the healthcare session.

11. A computer program product residing on a computer readable medium having a plurality of instructions stored thereon which, when executed by a processor, cause the processor to perform operations comprising:
monitoring a conversation between a plurality of participants of a telehealth session;
identifying an addressable issue within the conversation; and
initiating an action to mitigate the addressable issue.

12. The computer program product of claim 11 wherein:
a first participant of the plurality of participants is a medical professional; and
a second participant of the plurality of participants is a patient.

13. The computer program product of claim 11 wherein the addressable issue is a potential language barrier with one of the participants of the telehealth session.

14. The computer program product of claim 13 wherein initiating an action to mitigate the addressable issue includes:
translating audio received by one of the participants of the telehealth session from a first language to a second language.

15. The computer program product of claim 14 wherein translating audio received by one of the participants of the telehealth session from a first language to a second language includes:
providing a written translation of audio received by one of the participants of the telehealth session from the first language to the second language.

16. The computer program product of claim 14 wherein translating audio received by one of the participants of the telehealth session from a first language to a second language includes:
providing a verbal translation of audio received by one of the participants of the telehealth session from the first language to the second language.

17. The computer program product of claim 11 wherein the addressable issue is a potential lack of understanding with one of the participants of the telehealth session.

18. The computer program product of claim 17 wherein initiating an action to mitigate the addressable issue includes:
providing a text-based summary of at least a portion of the conversation to a specific participant of the telehealth session; and
requesting that the specific participant of the healthcare session confirm their understanding of the text-based summary.

19. The computer program product of claim 18 wherein the text-based summary is a text-based summary of at least a portion of the conversation provided by the specific participant of the healthcare session.

20. The computer program product of claim 18 wherein the text-based summary is a text-based summary of at least a portion of the conversation provided by another participant of the healthcare session.

21. A computing system including a processor and memory configured to perform operations comprising:
monitoring a conversation between a plurality of participants of a telehealth session;
identifying an addressable issue within the conversation; and
initiating an action to mitigate the addressable issue.

22. The computing system of claim 21 wherein:
a first participant of the plurality of participants is a medical professional; and
a second participant of the plurality of participants is a patient.

23. The computing system of claim 21 wherein the addressable issue is a potential language barrier with one of the participants of the telehealth session.

24. The computing system of claim 23 wherein initiating an action to mitigate the addressable issue includes:
translating audio received by one of the participants of the telehealth session from a first language to a second language.

25. The computing system of claim 24 wherein translating audio received by one of the participants of the telehealth session from a first language to a second language includes:
providing a written translation of audio received by one of the participants of the telehealth session from the first language to the second language.

26. The computing system of claim 24 wherein translating audio received by one of the participants of the telehealth session from a first language to a second language includes:
providing a verbal translation of audio received by one of the participants of the telehealth session from the first language to the second language.

27. The computing system of claim 21 wherein the addressable issue is a potential lack of understanding with one of the participants of the telehealth session.

28. The computing system of claim 27 wherein initiating an action to mitigate the addressable issue includes:
providing a text-based summary of at least a portion of the conversation to a specific participant of the telehealth session; and
requesting that the specific participant of the healthcare session confirm their understanding of the text-based summary.

29. The computing system of claim 28 wherein the text-based summary is a text-based summary of at least a portion of the conversation provided by the specific participant of the healthcare session.

30. The computing system of claim 28 wherein the text-based summary is a text-based summary of at least a portion of the conversation provided by another participant of the healthcare session.
